(19) United States
(12) Patent Application Publication
(10) Pub. No.: US 2023/0054269 A1
(43) Pub. Date: Feb. 23, 2023

(54) Basket Catheter with Porous Sheath

(71) Applicant: Biosense Webster (Israel) Ltd., Yokneam (IL)

(72) Inventors: Assaf Govari, Haifa (IL); Andres Claudio Altmann, Haifa (IL); Christopher Thomas Beeckler, Brea, CA (US); Joseph Thomas Keyes, Sierra Madre, CA (US); Justin George Lichter, Irvine, CA (US)

(21) Appl. No.: 17/887,201

(22) Filed: Aug. 12, 2022

Related U.S. Application Data
(60) Provisional application No. 63/236,135, filed on Aug. 23, 2021.
(60) Provisional application No. 63/274,334, filed on Nov. 1, 2021.

(51) Int. Cl. A61B 18/14; A61B 18/12

(52) U.S. Cl. CPC A61B 18/1492; A61B 18/1206; A61B 2018/00077

(57) ABSTRACT

Medical apparatus includes an insertion tube configured for insertion into a body cavity of a patient and an expandable assembly connected distally to the insertion tube and comprising electrodes, which are configured to apply electrical energy to tissue within the body cavity. A flexible porous sheath is fitted over the expandable assembly and configured to contact the tissue within the body cavity so that the electrical energy is applied from the electrodes through the sheath to the tissue.

### CROSS-REFERENCE TO RELATED APPLICATION

This application claims the benefit of U.S. Provisional Patent Application 63/236,135, filed Aug. 23, 2021, and of U.S. Provisional Patent Application 63/274,334, filed Nov. 1, 2021. Both of these related applications are incorporated herein by reference.

### FIELD OF THE DISCLOSURE

The present disclosure relates generally to invasive medical equipment, and particularly to apparatus for ablating tissue within the body and methods for producing and using such apparatus.

### BACKGROUND

Cardiac arrythmias are commonly treated by ablation of myocardial tissue in order to block arrhythmogenic electrical pathways. For this purpose, a catheter is inserted through the patient's vascular system into a chamber of the heart, and an electrode or electrodes at the distal end of the catheter are brought into contact with the tissue that is to be ablated. In some cases, high-power radio-frequency (RF) electrical energy is applied to the electrodes in order to ablate the tissue thermally. Alternatively, high-voltage pulses may be applied to the electrodes in order to ablate the tissue by irreversible electroporation (IRE).

Some ablation procedures use basket catheters, in which multiple electrodes are arrayed along the spines of an expandable assembly at the distal end of the catheter.

The spines bend outward to form a basket-like shape and contact tissue within a body cavity. For example, U.S. Patent Application Publication 2020/0289197 describes devices and methods for electroporation ablation therapy, with the device including a set of spines coupled to a catheter for medical ablation therapy. Each spine of the set of splines may include a set of electrodes formed on that spine. The set of spines may be configured for translation to transition between a first configuration and a second configuration.

The present disclosure will be more fully understood from the following detailed description of the examples thereof, taken together with the drawings in which:

### BRIEF DESCRIPTION OF THE DRAWINGS

FIG. 1 is a schematic pictorial illustration showing a system for cardiac ablation, in accordance with an embodiment of the invention;

FIG. 2 is a schematic side view of a catheter expandable assembly with a porous sheath, in accordance with an embodiment of the invention;

FIGS. 3A and 3B are schematic cutaway views of the catheter expandable assembly of FIG. 2 in collapsed and expanded configurations, respectively, in accordance with an embodiment of the invention;

FIG. 4 is a flow chart that schematically illustrates a method for producing a sheath for a catheter expandable assembly, in accordance with an embodiment of the invention;

FIG. 5 is a schematic side view of a system for producing sheaths for catheter basket assemblies, in accordance with an embodiment of the invention;

FIG. 6 is a schematic side view of a braided tube produced using the system of FIG. 5, in accordance with an embodiment of the invention;

FIG. 7A is a side view of an exemplary expandable member; and

FIG. 7B is a side view of yet another expandable member that can be used with the sheaths produced from the braided tube of FIG. 6.

### DETAILED DESCRIPTION OF EXAMPLES

Catheters with expandable assemblies, such as basket catheters, are useful in performing ablation procedures rapidly and efficiently, since the spines of the basket catheter (and thus the electrodes on the spines) are able to contact and ablate the tissue at multiple locations concurrently. The spines themselves, however, can give rise to dangerous blood clots during the ablation procedure, due to the disturbance they cause in the blood flow, as well as due to arcing between the spines, particularly in IRE-based ablation. Furthermore, a spine can become embedded in the tissue during the procedure, which can lead to local overheating, resulting in charring and/or other trauma. The use of spines having smooth, rounded profiles can be helpful in mitigating these effects, but by itself does not eliminate the problems of clotting and tissue damage.

Examples of the present disclosure that are described herein address these problems by covering the expandable assembly with a porous sheath. (As used herein, the term “sheath” is intended to include “an outer cover” or a “membrane.”) The sheath prevents direct contact between the spines and the tissue, while still permitting electrical energy to be applied from the electrodes through the sheath to the tissue. The type of material and thickness of the sheath may be chosen so that irrigation fluid delivered through the catheter to the expandable assembly can pass outward through the sheath to the tissue, while still preventing blood from penetrating inward through the sheath from the body cavity. The sheath is thus useful in preventing both clotting and tissue damage.

Based on these principles, the disclosed examples provide medical apparatus comprising an insertion tube for insertion into a body cavity of a patient and an expandable assembly connected distally to the insertion tube. A flexible porous sheath is fitted over the expandable assembly so that the sheath contacts the tissue within the body cavity. The expandable assembly comprises electrodes, which apply electrical energy through the sheath to tissue within the body cavity. Although the examples that are described hereinbelow relate specifically to a basket catheter for intracardiac ablation, the principles of the present disclosure may be adapted for use in other sorts of procedures in which electrical energy is applied to biological tissues.

In some examples, an electrical signal generator applies electrical energy to the electrodes on the expandable assembly with an amplitude sufficient to ablate the tissue contacted by the spines. In one example, the electrical signal generator applies bipolar electrical pulses to the electrodes with an amplitude sufficient so that the electrical energy applied from the electrodes through the sheath causes irreversible electroporation (IRE) in the tissue. Additionally or alternatively, the electrical signal generator applies a radio-frequency (RF) current to the electrodes with a power sufficient so that the electrical energy applied from the electrodes through the sheath causes thermal ablation of the tissue.

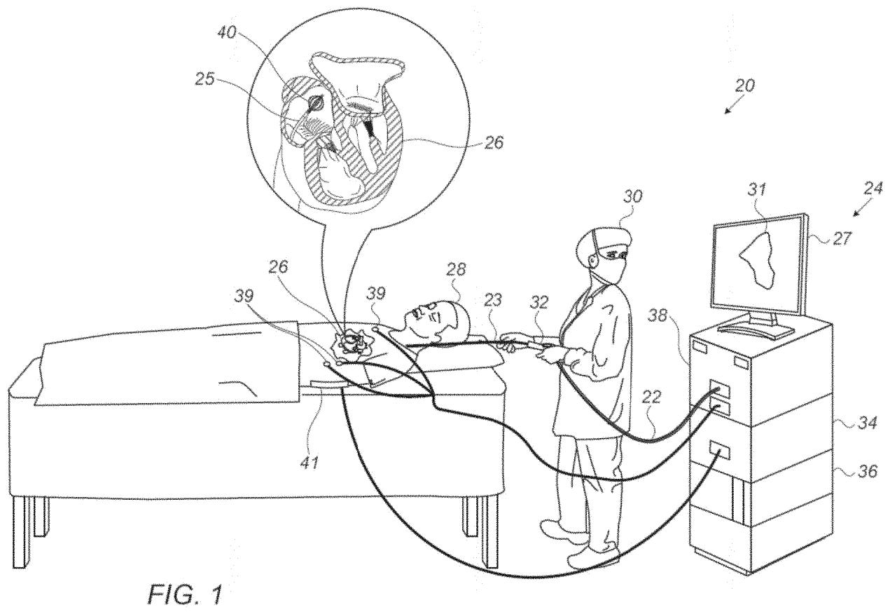

FIG. 1 is a schematic pictorial illustration of a system 20 used in an ablation procedure, in accordance with an example of the disclosure. Elements of system 20 may be based on components of the CARTO® system, produced by Biosense Webster, Inc. (Irvine, Calif).

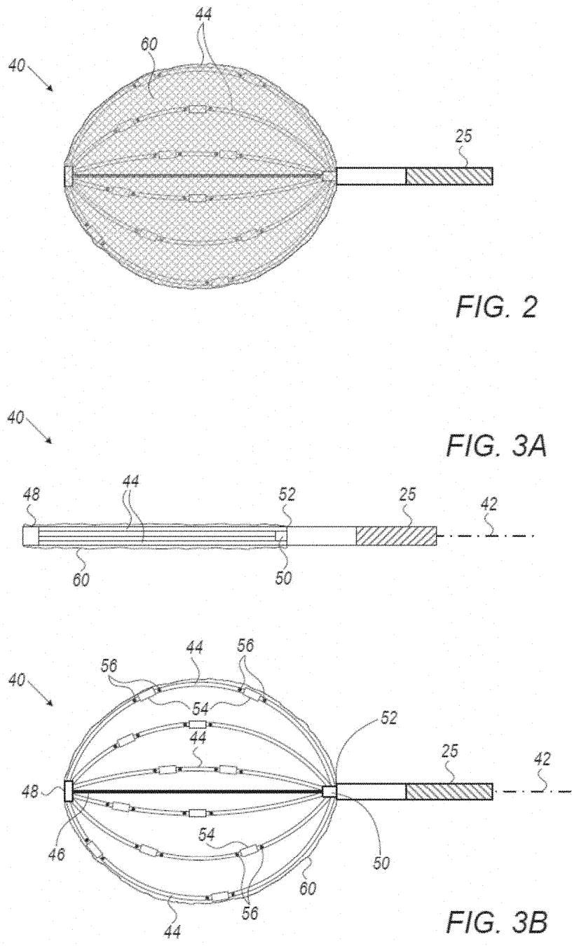

A physician 30 navigates a catheter 22 through the vascular system of a patient 28 into a chamber of a heart 26 of the patient, and then deploys an expandable assembly, such as a basket assembly 40, over which a flexible porous sheath is fitted (as shown in detail in FIGS. 2 and 3A/B), at the distal end of the catheter. The proximal end of basket assembly 40 is connected to the distal end of an insertion tube 25, which physician 30 steers using a manipulator 32 near the proximal end of catheter 22. Basket assembly 40 is inserted in a collapsed configuration through a tubular delivery sheath 23, which passes through the vascular system of patient 28 into the heart chamber where the ablation procedure is to be performed. Once inserted into the heart chamber, basket assembly 40 is deployed from the tubular sheath and allowed to expand within the chamber. Catheter 22 is connected at its proximal end to a control console 24. A display 27 on console 24 may present a map 31 or other image of the heart chamber with an icon showing the location of basket assembly 40 in order to assist physician 30 in positioning the basket assembly at the target location for the ablation procedure.

Once basket assembly 40 is properly deployed and positioned in heart 26, physician 30 actuates an electrical signal generator 38 in console 24 to apply electrical energy (such as IRE pulses or RF waveforms) to the electrodes on the basket assembly, under the control of a processor 36. The electrical energy may be applied in a bipolar mode, between pairs of the electrodes on basket assembly 40, or in a unipolar mode, between the electrodes on basket assembly 40 and a separate common electrode, for example a conductive back patch 41, which is applied to the patient's skin. During the ablation procedure, an irrigation pump 34 delivers an irrigation fluid, such as normal saline solution, through insertion tube 25 to basket assembly 40.

Typically, catheter 22 comprises one or more position sensors (not shown in the figures), which output position signals that are indicative of the position (location and orientation) of basket assembly 40. For example, basket assembly 40 may incorporate one or more magnetic sensors, which output electrical signals in response to an applied magnetic field. Processor 36 receives and processes the signals in order to find the location and orientation coordinates of basket assembly 40, using techniques that are known in the art and are implemented, for example, in the above-mentioned Carto system. Alternatively or additionally, system 20 may apply other position-sensing technologies in order to find the coordinates of basket assembly 40. For example, processor 36 may sense the impedances between the electrodes on basket assembly 40 and body-surface electrodes 39, which are applied to the chest of patient 28, and may convert the impedances into location coordinates using techniques that are likewise known in the art. In any case, processor 36 uses the coordinates in displaying the location of basket assembly 40 on map 31.

Alternatively, catheter 22 and the ablation techniques that are described herein may be used without the benefit of position sensing. In such examples, for example, fluoroscopy and/or other imaging techniques may be used to ascertain the location of basket assembly 40 in heart 26.

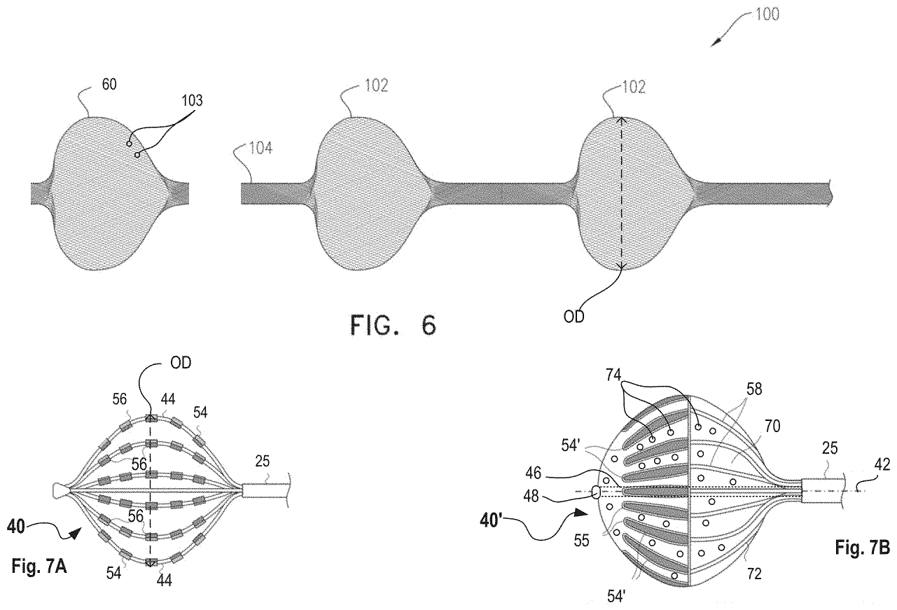

The system configuration that is shown in FIG. 1 is presented by way of example for conceptual clarity in understanding the operation of examples of the present disclosure. For the sake of simplicity, FIG. 1 shows only the elements of system 20 that are specifically related to an expandable assembly, such as basket assembly 40, and ablation procedures using the basket assembly. (Other expandable assemblies to which the principles of the present disclosure may be applied are described hereinbelow with reference to FIGS. 7A and 7B.) The remaining elements of the system will be apparent to those skilled in the art, who will likewise understand that the principles of the present disclosure may be implemented in other medical therapeutic systems, using other components. All such alternative implementations are considered to be within the scope of the present disclosure.

Reference is now made to FIGS. 2, 3A and 3B, which schematically show details of basket assembly 40, which is covered by a flexible, porous sheath 60 in accordance with an example of the disclosure. FIG. 2 is a side view of basket assembly 40 in its expanded state, while FIGS. 3A and 3B are cutaway views showing the basket assembly in collapsed and expanded states, respectively.

Basket assembly 40 has a distal end 48 and a proximal end 50, which is connected to a distal end 52 of insertion tube 25. The basket assembly comprises multiple spines 44, whose proximal ends are conjoined at proximal end 50, and whose distal ends are conjoined at distal end 48. One or more electrodes 54 are disposed externally on each of spines 44. Alternatively, spines 44 may comprise a solid conducting material and may thus serve as electrodes themselves, for example as described in U.S. patent application Ser. No. 16/842,648, filed Apr. 7, 2020, whose disclosure is incorporated herein by reference.

Irrigation outlets 56 in spines 44 allow irrigation fluid flowing within the spines to exit and irrigate tissue in the vicinity of electrodes 54. Alternatively or additionally, the irrigation outlets may be located elsewhere in the basket assembly, for example on an irrigation manifold that is contained inside the basket assembly (not shown in the figures).

Sheath 60 is fitted over basket assembly 40 and thus contacts the tissue in heart 26 when the basket assembly is expanded and advanced against the tissue. Sheath 60 prevents direct contact between spines 44 and the heart tissue. Thus, the electrical energy that is applied to electrodes 54 passes through sheath 60 to the tissue. In one example, sheath 60 comprises expanded polytetrafluoroethylene (ePTFE), for example with a thickness of about 70 μm. The ePTFE sheath is advantageous in being lubricious, smooth, strong, and biocompatible and in preventing spines 44 from becoming embedded in the heart tissue.

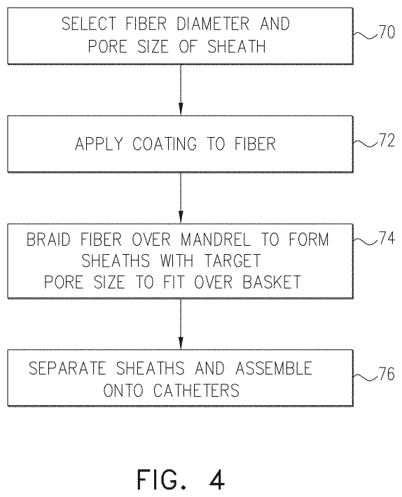

Alternatively, sheath 60 comprises a tube made by braiding suitable polymer fibers, such as a polyethylene terephthalate (PET) or polyamide (nylon) yarn. The tube may be braided with a variable diameter so as to conform better to the deployed basket shape. Specifically, the proximal diameter of the tube may be made to fit the proximal neck of basket, and the distal diameter may be made as small as possible. The distal end may be closed by fastening the loose yarn ends with an adhesive, melting the yarn ends together, or any other suitable method of sealing. An advantage of utilizing a fabric in a tubular shape rather than a flat shape is that the material better conforms to the basket shape, and pleats are avoided or minimized. Avoidance of pleats is helpful in reducing the collapsed diameter of sheath 60 and also reduces the potential for blood to coagulate in the folds of the material. A process for production of this sort of braided sheath is described further hereinbelow with reference to FIGS. 4-6.

In yet another example, sheath 60 is made from a sheet of flexible, non-porous material, and pores of the desired size are drilled through the material, for example by laser drilling. In a further example, sheath 60 can be formed by blow-molding a smaller tubular member to form a balloon membrane, with pores subsequently formed through the balloon membrane by laser drilling.

The pores in sheath 60 are sufficiently large to permit the irrigation fluid to pass from irrigation outlets 56 outward through sheath 60 to irrigate the heart tissue, while preventing blood from penetrating inward through the sheath from the heart chamber. The inventors have found it advantageous for this purpose that the pores in the sheath have areas between 10 μm² and 100,000 μm². The best results were obtained with pores having areas between 100 μm² and 10,000 μm². These ranges of pore areas are also useful in ensuring that the electrical energy from electrodes 54 passes freely out through sheath 60 to the adjoining tissue in order to ablate the tissue. Specifically, these ranges of pore areas ensure that irrigation fluid (which is electroconductive) can flow from inside sheath 60 through the pores to the tissue outside the sheath.

The polymer fibers that are used in producing sheath 60, such as PET and nylon fibers, are inherently insulators. Both PET and nylon, however, are hygroscopic, and once the fibers absorb water (or irrigation fluid), they become more conductive and thus enable the electrical energy output by electrodes 54 to pass more freely through sheath 60 to the target tissue. To enhance the performance of sheath 60 in this respect, in one example the polymer fibers are coated with a hydrophilic material. The hydrophilic coating attracts water into the fibers, so that sheath 60 becomes more conductive and thus facilitates efficient ablation. The coating also makes the sheath more lubricious, reducing the force necessary to collapse the basket.

In an alternative example, a hydrophobic coating is applied to the polymer fibers of the sheath. The hydrophobic coating requires the sheath to be pressurized in order for irrigation fluid to flow through it. This positive pressure prevents blood from entering the sheath even when the irrigation is at a low flow rate.

In the collapsed state of FIG. 3A, spines 44 are straight and aligned parallel to a longitudinal axis 42 of insertion tube 25, to facilitate insertion of basket assembly 40 into heart 26. In this state, sheath 60 collapses inward together with spines 44. To ensure that sheath 60 will collapse with basket assembly 40, sheath 60 is joined to distal end 48 and proximal end 50 of balloon assembly 40. Upon extension of an actuator 46 to separate distal end 48 from proximal end 50, both sheath 60 and spines 44 will compress into a tubular profile of FIG. 3A. Upon retraction of actuator 46 toward proximal end 50, spines 44 and sheath 60 will expand into the spherical like configuration shown in FIG. 3B. In the expanded state of FIG. 3B, spines 44 bow radially outward, causing sheath 60 to expand and contact tissue within the heart.

In one example, spines 44 are produced such that the stable state of basket assembly 40 is the collapsed state of FIG. 3A: In this case, when basket assembly 40 is pushed out of the sheath, it is expanded by drawing actuator 46, such as a suitable wire, in the proximal direction through insertion tube 25. Releasing actuator 46 allows basket assembly 40 to collapse back to its collapsed state.

In another example, spines 44 are produced such that the stable state of basket assembly 40 is the expanded state of FIG. 3B: In this case, basket assembly 40 opens out into the expanded state when it is pushed out of the sheath, and actuator 46 may be replaced by a flexible pusher rod for straightening spines 44 before withdrawing the basket assembly back into the sheath.

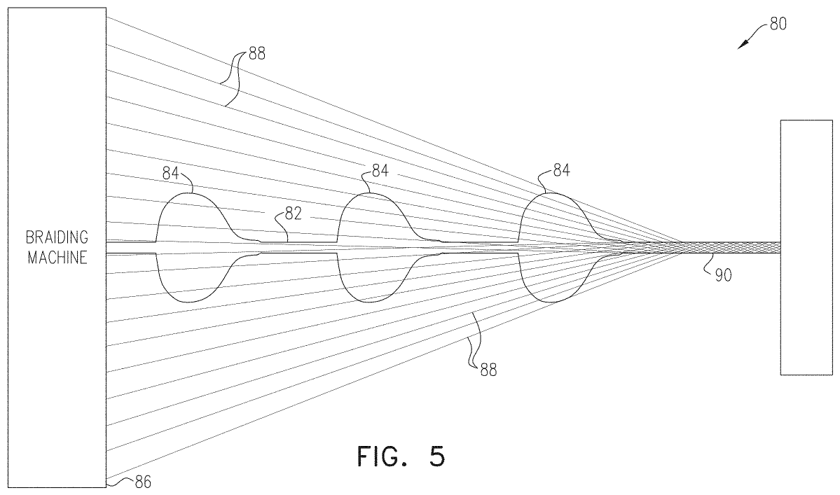

Reference is now made to FIGS. 4-6, which schematically illustrate a method for producing sheaths 60 for a catheter basket assembly, in accordance with an example of the disclosure. FIG. 4 is a flow chart showing steps in the method, while FIG. 5 is a schematic side view of a system 80 for producing the sheaths. FIG. 6 is a schematic side view of a braided tube 100 produced using the system of FIG. 5.

As a preliminary step, the diameter of fibers 88 that are to be used in producing the sheaths and the sizes of the pores to be formed in the sheaths are selected, at a fiber selection step 70. For example, PET or nylon fibers of approximately 25 to 100 denier may be used, and the pores in the sheath may have areas from approximately 10 μm² to approximately 100,000 μm², as noted above. If desired, a hydrophilic or hydrophobic coating may be applied to the fibers, at a coating step 72.

Fibers 80 are braided over a suitable mandrel 90 to form a tube 100 having a varying diameter, at a braiding step 74. As shown in FIG. 5, mandrel 90 comprises multiple bulbous protrusions 84 disposed along a shaft 82. In one example, bulbous protrusions 84 can comprise a balloon member inflated to a desired shape so that it serves as an underlying support structure for the braiding of the fibers. A braiding machine 86, as is known in the art, braids fibers 88 over mandrel 90. The resulting tube 100, as shown in FIG. 6, comprises bulbs 102 of the desired size, with narrower necks 104 in between. Bulbs 102 are sized to fit over basket assemblies 40, while necks 104 fit snugly over the distal end of insertion tube 25 (as shown in FIG. 2). The braiding parameters of braiding machine 86 are set so that bulbs 102 contain openings (pores 103) of the desired size (for example, of a desired diameter or area).

To ensure firm contact between sheath 60 and the expandable assembly over which it is to fit, such as basket assembly 40, sheath 60 can be sized to be slightly smaller than the expandable assembly. For example, each bulb 102 (defining the inner diameter of sheath 60) can be sized such that the maximum outer diameter of the bulb 102 is approximately 5% to 20% smaller than the maximum outer diameter (OD) of the expandable assembly over which it is to fit. The maximum OD of the expandable assembly can be measured from the radially outermost points of the expandable assembly (e.g., from one electrode to diametrically opposed electrode or from one spine to a diametrically opposed spine.)

Necks 104 in tube 100 are cut to separate the tube into multiple separate bulbs 102, each of which now becomes a sheath 60, at a sheath separation step 76. Prior to the separation of bulbs 102 into individual sheaths 60, the underlying bulbous protrusions 84 are deflated (if inflated previously) and withdrawn through tube 100. Alternatively, bulbous protrusions 84 may be withdrawn after separation of bulbs 102 into separate pieces. As noted earlier, the distal ends of bulbs 102 are closed after cutting by fastening together the loose ends of fibers 88 with an adhesive, melting the ends together, or any other suitable method of sealing. Sheaths 60 are then fitted over basket assemblies 40.

FIG. 7A and 7B are side views of exemplary expandable members 40 and 40′, respectively, which can be used with the sheaths produced from braided tubes as described above. Sheaths 60 (formerly bulbs 102) are fitted over expandable assemblies 40 or 40′ by compressing expandable assembly 40 (FIG. 7A) or deflating assembly 40′ (FIG. 7B) so that assembly 40 or 40′ will fit within the smaller tubular part of sheath 60 (e.g., neck 104).

In the example of FIG. 7B, expandable assembly 40′ is in the form of a balloon membrane 70 coupled to a distal end of insertion tube 25. A plurality of electrodes 54′ can be disposed on respective substrates 55 disposed on the outer surface of membrane 70. Conductive members 72 can be used to deliver electrical energy to respective electrodes 54′. Conductive members 72 can be disposed inside or outside membrane 70 in the form of electrical traces. Alternatively, conductive members 72 can be wires disposed within the internal volume defined by balloon membrane 70. Conductive members 72 can extend through insertion tube 25 all the way to console 24. Irrigation pores 74 extend through balloon membrane 70 to allow irrigation fluid delivered from insertion tube 25 to flow through membrane 70 and through pores 103 of sheath 60. An actuator 46 (shown by dashed lines) can be mounted inside membrane 70 so that actuator 46 is fixed to distal end 48 and allows for extension of distal end 48 relative to insertion tube 25 (i.e., compressing membrane 70 into a smaller outer profile) or retraction of distal end 48 relative to insertion tube 25 (i.e., causing expansion of membrane 70 into a larger profile). Membrane 70 is typically made of a less flexible material than porous covering 60.

Assembly of expandable member 40′ can be completed by deflating membrane 70 and inserting member 40′ into the smaller tube (e.g., neck 104) of sheath 60. Thereafter, member 40′ can be inflated, and the ends of sheath 60 can be joined to the proximal and distal end of membrane 70. Details of an expandable member 40′ of this sort are described in U.S. Patent Application Publication 2021/0169567, which is hereby incorporated by reference as if set forth herein.

### EXAMPLES

Example 1: Medical apparatus (20), comprising an insertion tube (25) configured for insertion into a body cavity of a patient and an expandable assembly (40) connected distally to the insertion tube and comprising electrodes (54), which are configured to apply electrical energy to tissue within the body cavity. A flexible porous sheath (60) is fitted over the expandable assembly and configured to contact the tissue within the body cavity so that the electrical energy is applied from the electrodes through the sheath to the tissue.

Example 2: The apparatus according to example 1, wherein the sheath comprises expanded polytetrafluoroethylene (ePTFE).

Example 3. The apparatus according to example 1, wherein the sheath comprises a braided polymer fiber.

Example 4. The apparatus according to example 3, wherein the braided polymer fiber comprises polyethylene terephthalate (PET).

Example 5: The apparatus according to example 3, wherein the braided polymer fiber comprises polyamide.

Example 6: The apparatus according to example 3, wherein the sheath is braided as a tube of varying diameter.

Example 7: The apparatus according to example 1, wherein the sheath comprises a polymer fiber having a hydrophilic coating.

Example 8: The apparatus according to example 1, wherein the sheath comprises a polymer fiber having a hydrophobic coating.#

Example 9: The apparatus according to example 1, wherein the expandable assembly comprises one or more irrigation outlets, which are coupled to convey an irrigation fluid from the insertion tube to the tissue through the sheath.

Example 10: The apparatus according to example 9, wherein the sheath comprises a fabric chosen to permit the irrigation fluid to pass outward through the sheath from the one or more irrigation outlets to the tissue while preventing blood from penetrating inward through the sheath from the body cavity.

Example 11: The apparatus according to example 10, wherein the porous sheath contains pores having respective areas between 10 μm² and 100,000 μm².

Example 12: The apparatus according to example 11, wherein the respective areas of the pores are between 100 μm² and 10,000 μm².

Example 13: The apparatus according to example 9, and comprising an irrigation pump, which is coupled to supply the irrigation fluid to the insertion tube for conveyance to the irrigation outlets.

Example 14: The apparatus according to example 1, wherein the expandable assembly comprises a plurality of resilient spines, having respective proximal and distal tips, wherein the proximal tips of the spines are joined mechanically at a proximal end of the expandable assembly, and the distal tips of the spines are joined mechanically at a distal end of the expandable assembly, and the spines bow radially outward when the expandable assembly is deployed in the body cavity, thereby causing the sheath to contact the tissue in the body cavity.

Example 15: The apparatus according to example 14, wherein the spines comprise a conductive material, which is configured to serve as an electrode.

Example 16: The apparatus according to example 14, wherein the spines are configured to collapse radially inward so that the spines are aligned along an axis of the insertion tube while the apparatus is being inserted into the body cavity.

Example 17: The apparatus according to example 1, and comprising an electrical signal generator configured to apply electrical energy to the electrodes with an amplitude sufficient to ablate the tissue.

Example 18: The apparatus according to example 17, wherein the electrical signal generator is configured to apply bipolar electrical pulses to the electrodes with an amplitude sufficient so that the electrical energy applied from the electrodes through the sheath causes irreversible electroporation (IRE) in the tissue.

Example 19: The apparatus according to example 17, wherein the electrical signal generator is configured to apply a radio-frequency (RF) current to the electrodes with a power sufficient so that the electrical energy applied from the electrodes through the sheath causes thermal ablation of the tissue.

Example 20: The apparatus according to claim 1, wherein the expandable assembly comprises a balloon membrane having an outer surface on which the electrodes are disposed, each of the plurality of electrodes being electrically connected to at least one respective conductive member extending through the insertion tube, wherein irrigation pores extend through the balloon membrane to allow irrigation fluid to flow from the insertion tube through the irrigation pores.

Example 21: A method for producing a medical device comprises providing an insertion tube (25) configured for insertion into a body cavity of a patient and connecting distally to the insertion tube a expandable assembly (40) comprising electrodes (54). A flexible porous sheath (60) is fitted over the expandable assembly so that the sheath contacts tissue within the body cavity when the insertion tube is inserted into the body cavity.

Example 22: The method according to example 21, wherein fitting the flexible porous sheath comprises braiding a polymer fiber to form the sheath.

Example 23: The method according to example 22, wherein braiding the polymer fiber comprises braiding a tube with a varying diameter.

Example 24: The method according to example 23, wherein braiding the tube comprises braiding polymer fibers over a mandrel comprising multiple bulbous protrusions disposed along a shaft, and cutting the braided tube to form multiple sheaths.

Example 25: The method according to example 22, and comprising applying a hydrophilic coating to the polymer fiber.

Example 26: The method according to example 22, and comprising applying a hydrophobic coating to the polymer fiber.

Various features of the disclosure which are, for clarity, described in the contexts of separate examples may also be provided in combination in a single example. Conversely, various features of the disclosure which are, for brevity, described in the context of a single example may also be provided separately or in any suitable sub-combination.#

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. Medical apparatus, comprising:
an insertion tube configured for insertion into a body cavity of a patient;
an expandable assembly connected distally to the insertion tube and comprising electrodes, which are configured to apply electrical energy to tissue within the body cavity; and
a flexible porous sheath, which is fitted over the expandable assembly and configured to contact the tissue within the body cavity so that the electrical energy is applied from the electrodes through the sheath to the tissue.

2. The apparatus according to claim 1, wherein the sheath comprises expanded polytetrafluoroethylene (ePTFE).

3. The apparatus according to claim 1, wherein the sheath comprises a braided polymer fiber.

4. The apparatus according to claim 3, wherein the braided polymer fiber comprises polyethylene terephthalate (PET).

5. The apparatus according to claim 3, wherein the braided polymer fiber comprises polyamide.

6. The apparatus according to claim 3, wherein the sheath is braided as a tube of varying diameter.

7. The apparatus according to claim 1, wherein the sheath comprises a polymer fiber having a hydrophilic coating.

8. The apparatus according to claim 1, wherein the sheath comprises a polymer fiber having a hydrophobic coating.

9. The apparatus according to claim 1, wherein the expandable assembly comprises one or more irrigation outlets, which are coupled to convey an irrigation fluid from the insertion tube to the tissue through the sheath.

10. The apparatus according to claim 9, wherein the sheath comprises a fabric chosen to permit the irrigation fluid to pass outward through the sheath from the one or more irrigation outlets to the tissue while preventing blood from penetrating inward through the sheath from the body cavity.

11. The apparatus according to claim 10, wherein the porous sheath contains pores having respective areas between 10 μm² and 100,000 μm².

12. The apparatus according to claim 11, wherein the respective areas of the pores are between 100 μm² and 10,000 μm².

13. The apparatus according to claim 9, and comprising an irrigation pump, which is coupled to supply the irrigation fluid to the insertion tube for conveyance to the irrigation outlets.

14. The apparatus according to claim 1, wherein the expandable assembly comprises a plurality of resilient spines, having respective proximal and distal tips, wherein the proximal tips of the spines are joined mechanically at a proximal end of the expandable assembly, and the distal tips of the spines are joined mechanically at a distal end of the expandable assembly, and the spines bow radially outward when the expandable assembly is deployed in the body cavity, thereby causing the sheath to contact the tissue in the body cavity.

15. The apparatus according to claim 14, wherein the spines comprise a conductive material, which is configured to serve as an electrode.

16. The apparatus according to claim 14, wherein the spines are configured to collapse radially inward so that the spines are aligned along an axis of the insertion tube while the apparatus is being inserted into the body cavity.

17. The apparatus according to claim 1, and comprising an electrical signal generator configured to apply electrical energy to the electrodes with an amplitude sufficient to ablate the tissue.

18. The apparatus according to claim 17, wherein the electrical signal generator is configured to apply bipolar electrical pulses to the electrodes with an amplitude sufficient so that the electrical energy applied from the electrodes through the sheath causes irreversible electroporation (IRE) in the tissue.

19. The apparatus according to claim 17, wherein the electrical signal generator is configured to apply a radio-frequency (RF) current to the electrodes with a power sufficient so that the electrical energy applied from the electrodes through the sheath causes thermal ablation of the tissue.

20. The apparatus according to claim 1, wherein the expandable assembly comprises a balloon membrane having an outer surface on which the electrodes are disposed, each of the plurality of electrodes being electrically connected to at least one respective conductive member extending through the insertion tube, wherein irrigation pores extend through the balloon membrane to allow irrigation fluid to flow from the insertion tube through the irrigation pores.

21. A method for producing a medical device, comprising:
providing an insertion tube configured for insertion into a body cavity of a patient; #
connecting distally to the insertion tube an expandable assembly comprising electrodes; and
fitting a flexible porous sheath over the expandable assembly so that the sheath contacts tissue within the body cavity when the insertion tube is inserted into the body cavity.

22. The method according to claim 21, wherein the sheath comprises expanded polytetrafluoroethylene (ePTFE).

23. The method according to claim 21, wherein fitting the flexible porous sheath comprises braiding a polymer fiber to form the sheath.

24. The method according to claim 23, wherein the polymer fiber comprises polyethylene terephthalate (PET).

25. The method according to claim 23, wherein the polymer fiber comprises polyamide.

26. The method according to claim 23, wherein braiding the polymer fiber comprises braiding a tube with a varying diameter.

27. The method according to claim 26, wherein braiding the tube comprises braiding polymer fibers over a mandrel comprising multiple bulbous protrusions disposed along a shaft, and cutting the braided tube to form multiple sheaths.

28. The method according to claim 23, and comprising applying a hydrophilic coating to the polymer fiber.

29. The method according to claim 23, and comprising applying a hydrophobic coating to the polymer fiber.

30. The method according to claim 21, wherein the porous sheath contains pores having respective areas between 10 μm² and 100,000 μm².

31. The method according to claim 39, wherein the respective areas of the pores are between 100 μm² and 10,000 μm².

32. The method according to claim 21, and comprising conveying an irrigation fluid from the insertion tube to the tissue through the sheath.

33. The method according to claim 32, wherein the sheath comprises a fabric chosen to permit the irrigation fluid to pass outward through the sheath from the one or more irrigation outlets to the tissue while preventing blood from penetrating inward through the sheath from the body cavity.

34. The method according to claim 21, wherein connecting the expandable assembly comprises joining together respective distal tips of a plurality of resilient spines at a proximal end of the expandable assembly, and joining together respective distal tips of the spines at a distal end of the expandable assembly, so that the spines bow radially outward when the expandable assembly is deployed in the body cavity, thereby causing the sheath to contact the tissue in the body cavity.

35. The method according to claim 34, wherein the spines comprise a conductive material, which is configured to serve as an electrode.

36. The method according to claim 34, wherein the spines collapse radially inward so that the spines are aligned along an axis of the insertion tube while the expandable assembly is being inserted into the body cavity.

37. The method according to claim 21, and comprising coupling an electrical signal generator to apply electrical energy from the electrodes through the sheath to the tissue within the body cavity

38. The method according to claim 37, wherein coupling the electrical signal generator comprises applying the electrical energy to ablate the tissue within the body cavity.

39. The method according to claim 38, wherein applying the electrical energy comprises applying bipolar electrical pulses to the electrodes with an amplitude sufficient so that the electrical energy applied from the electrodes through the sheath causes irreversible electroporation (IRE) in the tissue.

39. The method according to claim 38, wherein applying the electrical energy comprises applying a radio-frequency (RF) current to the electrodes with a power sufficient so that the electrical energy applied from the electrodes through the sheath causes thermal ablation of the tissue.
